(19) United States
(12) Patent Application Publication
(10) Pub. No.: US 2018/0085159 A1
(43) Pub. Date: Mar. 29, 2018

(54) SELF STABILIZING ABLATION SUCTION CATHETER

(71) Applicant: Cardiac Pacemakers, Inc., St. Paul, MN (US)

(72) Inventors: Matthew Sulkin, New Brighton, MN (US); Jason J. Hamann, Blaine, MN (US); Jacob I. Laughner, St. Paul, MN (US); Mary M. Byron, Roseville, MN (US); Sarah R. Gutbrod, St. Paul, MN (US)

(21) Appl. No.: 15/702,928

(22) Filed: Sep. 13, 2017

Related U.S. Application Data
(60) Provisional application No. 62/400,550, filed on Sep. 27, 2016.

(51) Int. Cl. A61B 18/14

(52) U.S. Cl. CPC A61B 18/1492; A61B 2017/00053

(57) ABSTRACT

A catheter system includes a catheter and a control system. The catheter includes an elongate catheter body and a catheter tip coupled to a distal end of the elongate catheter body. The catheter tip includes a plurality of openings corresponding to a plurality of lumens extending through the elongate catheter body. The control system is configured to initiate a source of vacuum pressure to at least one of the plurality of lumens, receive an indication of a vacuum seal, and in response to the indication of the vacuum seal, initiate a source of ablation energy.

### CROSS-REFERENCE TO RELATED APPLICATION

This application claims priority to Provisional Application No. 62/400,550, filed Sep. 27, 2017, which is herein incorporated by reference in its entirety.

### TECHNICAL FIELD

The present disclosure relates generally to medical devices and, more particularly, to systems, devices and methods related to catheters used to perform ablation functions.

### BACKGROUND

Cardiac ablation is a procedure by which cardiac tissue is treated to inactivate the tissue. The tissue targeted for ablation may be associated with improper electrical activity, for example. Cardiac ablation can lesion the tissue and prevent the tissue from improperly generating or conducting electrical signals.

### SUMMARY

In Example 1, a catheter system includes a catheter and a control system. The catheter includes an elongate catheter body and a catheter tip coupled to a distal end of the elongate catheter body. The catheter tip includes a plurality of openings corresponding to a plurality of lumens extending through the elongate catheter body. The control system is configured to initiate a source of vacuum pressure to at least one of the plurality of lumens, receive an indication of a vacuum seal, and in response to the indication of the vacuum seal, initiate a source of ablation energy.

In Example 2, the catheter system of Example 1, wherein the control system is configured to initiate the source of ablation in response to calculating that the vacuum seal is maintained for a predetermined period of time.

In Example 3, the catheter system of any of Examples 1-2, wherein the control system is further configured to receive an indication of contact of the catheter tip, and in response to receiving the indication of contact, initiate the source of vacuum pressure.

In Example 4, the catheter system of any of Examples 1-3, wherein the control system is further configured to receive an indication of a loss of a vacuum seal, and in response to receiving the indication of a loss of a vacuum seal, turn off the source of ablation energy.

In Example 5, the catheter system of any of Examples 1-3, wherein the control system is further configured to receive an indication of a loss of a vacuum seal, and in response to receiving the indication of a loss of a vacuum seal, initiate an alert.

In Example 6, the catheter system of any of claims 1-5, wherein the control system is configured to control a valve coupled to a first lumen and a second lumen, the control system further configured to cause the valve to fluidly couple the first lumen to the source of vacuum pressure, and cause the valve to fluidly couple the second lumen to a source of irrigation fluid.

In Example 7, the catheter system of any of Examples 1-6, wherein the control system is configured to control a valve coupled to a plurality of lumens, the control system further configured to cause the valve to fluidly couple only one of the plurality of lumens to the source of vacuum pressure.

In Example 8, the catheter system of any of the Examples 1-7, further comprising a sensor configured to detect a vacuum seal.

In Example 9, the catheter system of Example 8, wherein the vacuum seal is detected by one of a pressure change and a change in blood flow.

In Example 10, the catheter system of any of Examples 8-9, wherein the sensor is one of an optical sensor, a flow sensor, a pressure sensor, and an oxygen sensor.

In Example 11, the catheter system of Example 1, further comprising the vacuum source coupled to each of the plurality of lumens; and a valve configured to selectively fluidly couple the plurality lumens to the vacuum source.

In Example 12, the catheter system of Example 11, wherein the catheter includes a plurality of ablation electrodes.

In Example 13, the catheter system of Example 11, wherein the catheter includes a single ablation electrode.

In Example 14, a catheter system includes a catheter and a valve. The catheter includes an elongated catheter body, a first lumen and a second lumen extending through the elongated catheter body, and a catheter tip coupled to the elongated body. The catheter tip includes a first opening coupled to the first lumen and a second opening coupled to the second lumen. The valve is configured to fluidly couple the first lumen and the second lumen to a vacuum source.

In Example 15, the catheter system of Example 14, wherein the valve is further configured to fluidly couple the first lumen and the second lumen to an irrigation source.

In Example 16, a catheter system includes a catheter with an elongated catheter body, a first lumen extending through the elongated catheter body and configured to provide a vacuum, a second lumen extending through the elongated catheter body and configured to provide a vacuum, and a catheter tip coupled to the elongated body. The catheter tip includes a first opening coupled to the first lumen and configured to provide a vacuum seal and a second opening coupled to the second lumen and configured to provide a vacuum seal.

In Example 17, the catheter system of Example 16, further comprising a valve coupled to the first and second lumens, wherein the valve is configured to couple the first and second lumens to a vacuum source.

In Example 18, the catheter system of Example 17, wherein the valve is further configured to couple the first and second lumens to an irrigation fluid source.

In Example 19, the catheter system of Example 16, further comprising a third lumen extending through the elongated catheter body and configured to provide a vacuum, and wherein the catheter tip includes a third opening coupled to the third lumen and configured to provide a vacuum seal.

In Example 20, the catheter system of Example 16, wherein the catheter tip includes a plurality of ablation electrodes, and wherein the first opening is positioned between a pair of the plurality of ablation electrodes.

In Example 21, the catheter system of Example 16, wherein the catheter tip further includes a temperature sensor.

In Example 22, the catheter system of Example 21, wherein the catheter tip further includes at least one mapping sensor.

In Example 23, the catheter system of Example 16, wherein the first opening and the second opening have a surface area of 1-7.5 mm².

In Example 24, a catheter system includes control circuitry configured to: initiate a source of vacuum pressure to one or more lumens, receive an indication of a vacuum seal, and in response to the indication of the vacuum seal, initiate a source of ablation energy.

In Example 25, the catheter system of Example 24, wherein the control circuitry is configured to initiate the source of ablation in response to calculating that the vacuum seal is maintained for a predetermined period of time.

In Example 26, the catheter system of Example 24, wherein the control circuitry is further configured to receive an indication of contact of an ablation tip, and in response to receiving the indication of contact, initiate the source of vacuum pressure.

In Example 27, the catheter system of Example 24, wherein the control circuitry is further configured to receive an indication of a loss of a vacuum seal, and in response to receiving the indication of a loss of a vacuum seal, turn off the source of ablation energy.

In Example 28, the catheter system of Example 24, wherein the control circuitry is further configured to receive an indication of a loss of a vacuum seal, and in response to receiving the indication of a loss of a vacuum seal, initiate an alert.

In Example 29, the catheter system of Example 24, wherein the control circuitry is configured to control a valve coupled to a first lumen and a second lumen, the control system further configured to cause the valve to fluidly couple the first lumen to the source of vacuum pressure, and cause the valve to fluidly couple the second lumen to a source of irrigation fluid.

In Example 30, the catheter system of Example 24, wherein the control circuitry is configured to control a valve coupled to a plurality of lumens, the control system further configured to cause the valve to fluidly couple only one of the plurality of lumens to the source of vacuum pressure.

In Example 31, the catheter system of Example 24, further comprising a sensor configured to detect a vacuum seal.

In Example 32, the catheter system of Example 31, wherein the vacuum seal is detected by one of a pressure change and a change in blood flow.

In Example 33, the catheter system of Example 32, wherein the sensor is one of an optical sensor, a flow sensor, a pressure sensor, and an oxygen sensor.

In Example 34, the catheter system of Example 24, wherein the source of vacuum pressure is configured to provide at least 5 grams of force.

In Example 35, the catheter system of Example 24, wherein the control circuitry is configured to maintain the vacuum seal while ablation energy is applied.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows a catheter system, in accordance with certain embodiments of the present disclosure.

FIG. 2 shows a schematic side view of a portion of a catheter, in accordance with certain embodiments of the present disclosure.

FIG. 3 shows a cross-section view of the catheter of FIG. 2.

FIG. 4 outlines various steps of a routine, in accordance with certain embodiments of the present disclosure.

FIG. 5 shows a schematic side view of a portion of a catheter, in accordance with certain embodiments of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Various cardiac abnormalities can be attributed to improper electrical activity of cardiac tissue. Such improper electrical activity can include, but is not limited to, generation of electrical signals, conduction of electrical signals, and/or compression of the tissue in a manner that does not support efficient and/or effective cardiac function. For example, an area of cardiac tissue may become electrically active prematurely or otherwise out of synchrony during the cardiac cycle, causing the cardiac cells of the area and/or adjacent areas to contract out of rhythm. The result is an abnormal cardiac contraction that is not timed for optimal cardiac output. In some cases, an area of cardiac tissue may provide a faulty electrical pathway (e.g., a short circuit) that causes an arrhythmia, such as atrial fibrillation or supraventricular tachycardia. In some cases, inactive tissue (e.g., scar tissue) may be preferable to malfunctioning cardiac tissue.

Cardiac ablation is a procedure by which cardiac tissue is treated to inactivate the tissue. The tissue targeted for ablation may be associated with improper electrical activity, as described above. Cardiac ablation can lesion the tissue and prevent the tissue from improperly generating or conducting electrical signals. For example, a line, a circle, or other formation of ablated cardiac tissue can block the propagation of errant electrical signals. In some cases, cardiac ablation is intended to cause the death of cardiac tissue and to have scar tissue reform over the lesion, where the scar tissue is not associated with the improper electrical activity. Ablation therapies include radiofrequency (RF) ablation, cyroablation, microwave ablation, laser ablation, and surgical ablation, among others.

During an ablation procedure, an ablation tool such as a catheter with one or more ablation electrodes is advanced into contact with a target area of tissue where ablation energy (e.g., RF energy) is to be directed into the target tissue to form a lesion. Effective RF ablation relies on, among other things, maintaining contact with the tissue during the ablation procedure. Maintaining contact during a typical ablation cycle (e.g., 15-20 seconds) can be difficult to achieve because of a variety of reasons, including the fact that the heart continues to beat during the ablation procedure. Intermittent or unstable tissue contact results in RF energy being driven into blood surrounding the ablation electrode instead of the tissue. Features of the present disclosure are accordingly directed to catheter tip designs that assist with maintaining contact with tissue during ablation.

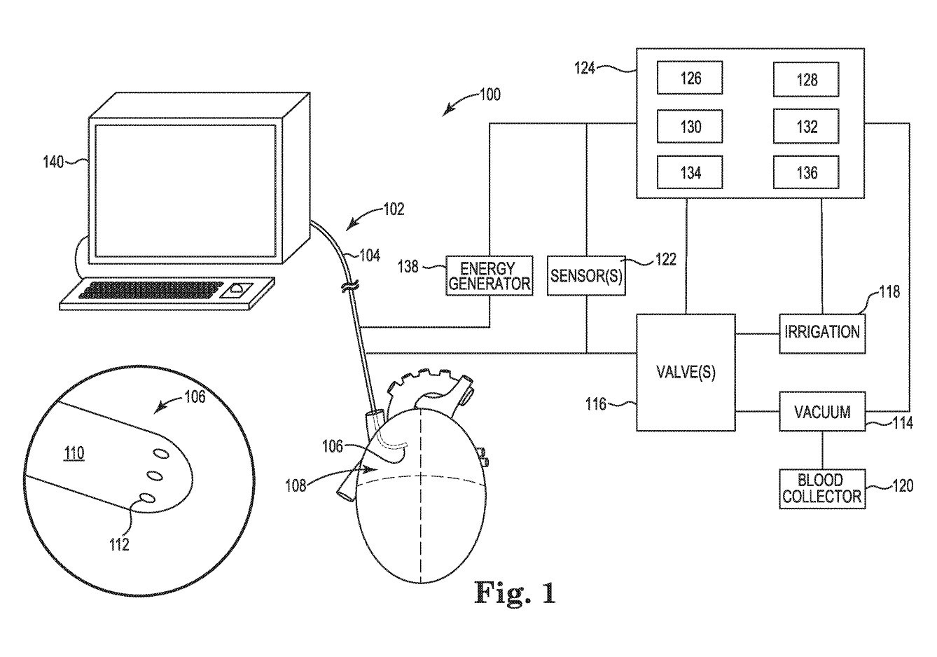

FIG. 1 shows a system 100 including a catheter 102 comprising an elongated catheter body 104 and a catheter tip 106, which is configured to be positioned within a heart 108. The catheter 102 includes an ablation electrode 110 coupled to the catheter tip 106. In operation, the ablation electrode 110 contacts targeted cardiac tissue to deliver ablative energy to the cardiac tissue, thus ablating the tissue to form a lesion, which can treat cardiac rhythm disturbances or abnormalities. The ablation electrode 110 in FIG. 1 is shown as radio frequency (RF) ablation electrode, which delivers RF energy to the cardiac tissue.

The catheter tip 106 includes openings 112 (three are shown in FIG. 1) each coupled to one or more lumens extending through the catheter 102. The lumens are coupled to a vacuum source 114 via one or more valves 116. The vacuum source 114 provides a negative pressure (e.g., suction, vacuum) to the lumens such that the catheter tip 106 (and therefore ablation electrode 110) can develop a vacuum seal at an interface between one or more of the openings 112 and thereby maintain contact between the catheter tip 106 and tissue. The vacuum source 114 can be various types of portable pumps and/or a wall-based vacuum source and the like.

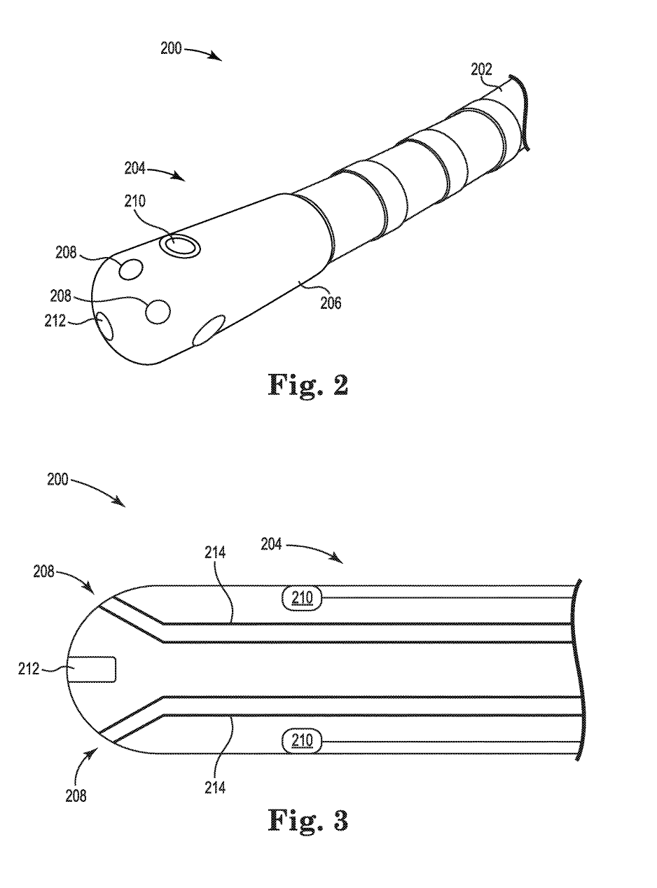

FIGS. 2-3 provide additional detail of a catheter 200 that can be used in the system 100. FIG. 2 shows a schematic, perspective view of the catheter 200, and FIG. 3 shows a cross-section view of the catheter 200. The catheter 200 includes an elongated catheter body 202 and a catheter tip 204. The catheter 200 also includes an ablation electrode 206 coupled to the catheter tip 204. The catheter 200 is shown as including at least two openings 208 positioned radially around the catheter tip 204—although fewer or more openings are contemplated. In some embodiments, the catheter 200 can include up to five openings that help maintain contact during ablation procedure while also allowing for space for other components at the catheter tip 204, such as mapping transducers 210 and temperature sensors 212. In some embodiments, the openings 208 are spaced equidistant from each other. Each opening 208 is shown as being coupled to an individual lumen 214 that extends from the opening 208 and through the catheter tip 206 and elongated catheter body 202. In some embodiments, multiple openings 208 can ultimately be coupled to a single lumen 214 that extends through the elongated catheter body 202.

The lumens 214 are coupled to a vacuum source (such as vacuum source 114 shown in FIG. 1) via the one or more valves 116. The vacuum source 114 provides a negative pressure (e.g., suction, vacuum) to one or more of the lumens 214 such that the catheter tip 204 (and therefore ablation electrode 206) can develop a vacuum seal at one or more of the openings 208 to maintain contact with tissue. It has been found that stable contact between a catheter and tissue can be secured and maintained by applying approximately 10 grams or more of suction force to the tissue. In some embodiments, the catheter is configured to provide at least 5 grams of suction force to the tissue. In some embodiments, the catheter is configured to provide 5-60 grams of suction force to the tissue. Using the contemplated suction force, the surface area of the openings 208 should range between 1-7.5 mm², which provides sufficient surface area for the catheter to “grasp” the tissue and maintain contact during an ablation procedure. Because the lumens 214 are subject to a negative pressure when coupled to the vacuum source 114 on one end of the lumen 214 and sealed against tissue on the other end of the lumen 214, the lumens 214 should be structurally strong to withstand collapsing. Example suitable materials include hard plastics and/or thermoplastics and the like.

The lumens 214 can also be coupled to an irrigation fluid source 118 via the one or more valves (such as valve 116 shown in FIG. 1). The valve 116 can include a manifold that controls which and/or whether the vacuum source 114 and irrigation fluid source 118 provide negative pressure or irrigation fluid, respectively, to one or more of the lumens 214. In some embodiments, the vacuum source 114 provides negative pressure to only one lumen 214 at a time. For example, to maintain contact with tissue, it may only be necessary to provide suction to the lumen 214 coupled to the opening 208 positioned closest to the tissue. Further, it may be difficult to ensure a vacuum seal at multiple openings, where a partially or unsealed opening with negative pressure would tend to suck blood through a lumen, which may be not desirable. The vacuum source 114 can be coupled to a blood collector 120, which receives blood transmitted through the lumens 214. The lumens 214 not providing suction can be utilized to pump irrigation fluid through to cool the ablation electrode 206, etc. In some embodiments, the vacuum source 114 provides negative pressure to multiple lumens 214.

In some embodiment, when the catheter tip 204 is determine to be near a target ablation site, the vacuum source 114 can be turned on to provide a negative pressure to one or more lumens 214. In some embodiments, the vacuum source 114 can be turned on upon determining that the catheter 200 is in contact with tissue. This can be determined using the one or more sensors 122 by detecting changes in impedance, capacitance, and the like. In some embodiments, the vacuum source 114 can be turned on and coupled to a particular lumen and therefore opening upon determining that a particular part or area of the catheter 200 is in contact with tissue.

Blood pulled into the one or more lumens 214 via the openings 208 would be collected at the blood collector 120. When the one or more openings 208 develop a vacuum seal with the tissue, a number of parameters could be detected by one or more sensors 122 to confirm contact and a seal. The one or more sensors 122 can include sensors that measure pressure, impedance, optical, oxygen, flow, and/or capacitance parameters. For example, when a vacuum seal is initiated, the sensor 122 could detect that blood flow (e.g., via parameters such as impedance, optical, capacitance, oxygen content, and the like) has decreased as a result of the vacuum seal. In another example, when a vacuum seal is initiated, the one or more sensors 122 could detect a pressure rise in the one or more lumens 214. The one or more sensors 122 can be coupled to the one or more lumens 214, for example, or coupled to other features of the system 100 including features external to the catheter 102. In some embodiments, the one or more sensors 122 are positioned within the one or more lumens 214.

The system 100 includes a control system 124 including a memory 126, a processor 128, a measurement sub-unit 130, a valve controller 132, a mapping sub-unit 134, and a display controller 136. The system 100 also includes an ablation energy generator 138 and a display 140. The control system 124 can be configured to carry out various routines, which may be carried out automatically or which may receive input or intervention from an operator of the system 100 at various stages of the routine.

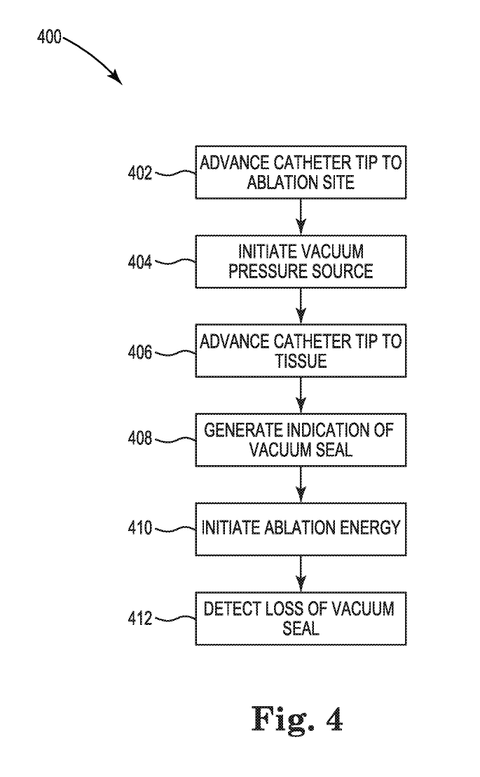

FIG. 4 provides an example routine 400 that includes some functions or steps that can be carried out by various components of the system 100, including the control system 124. During an ablation procedure, a catheter tip 106 is advanced near a target ablation site within a heart (step 402). Once the catheter tip 106 is in position, a negative pressure can be provided to one or more lumens. For example, the vacuum source 114 can be turned on and the valve 116 can be opened to provide a negative pressure to one or more lumens. In some embodiments, negative pressure is provided to one or more lumens in response to a catheter's contact detection system indicating contact between the catheter tip 106 and tissue. In some embodiments, only a particular lumen is supplied with a negative pressure. For example, based on information and/or images generated and displayed by the mapping sub-unit 134, display controller 136, and display 140, the control system 124 may only direct negative pressure to a lumen coupled to an opening 112 determined to be positioned closest to or already in contact with the tissue. The mapping sub-unit 134 receives mapping/positioning signals from mapping and/or navigation sensors coupled to the catheter 102 and determines physiological mapping and catheter position information. The display controller 136 outputs the results of the various sub-units to the display 140. For example, the display controller 136 can combine mapping, positioning information and output such information to the display 140, which can indicate which portions of a targeted ablation site are not fully ablated. Such information can be gathered and displayed in real-time to assist with monitoring and assessing lesion formation.

The vacuum source 114 can be turned on in response to input from an operator, which causes the control system 124 to initiate negative pressure from the vacuum source 114 (step 404). Likewise, the valve 116 can be opened in response to input from an operator that causes the control system 124 (e.g., valve controller 132) to initiate an open command to the valve 116. Once negative pressure is provided to the one or more lumens, blood may begin to be pulled into the lumens via the openings 112 and collected by the blood collector 120.

After a negative pressure is applied to the one or more lumens, the catheter tip 106 can be advanced towards to tissue if the catheter tip 106 is not already in contact with the tissue. Because of the negative pressure, when one or more of the openings 112 contact tissue, a vacuum seals develops—causing the catheter tip 106 to “grasp” the tissue and provide stable contact between the catheter tip 106 (and therefore ablation electrode 110) and tissue. When the one or more openings 112 develop a vacuum seal with the tissue, the one or more sensors can be used to detect one or more parameters that indicate the existence of a vacuum seal. For example, the one or more sensors 122 can measure parameters such as pressure, impedance, optical, oxygen, flow, and/or capacitance parameters. Once a given parameter exceeds or dips below a threshold, a signal indicating a vacuum seal can be generated (step 406). For example, the control system 124 (e.g., measurement sub-unit 130) can be configured to generate a signal in response to determining that a vacuum seal has developed between an opening and tissue. In some embodiments, a signal indicating a vacuum seal is generated after a vacuum seal is maintained for a predetermined amount of time.

In some embodiments, a signal indicating which lumen/opening has created a vacuum seal is generated. Such a signal can be used by the control system 124 to cause the valve 116 to remove application of a vacuum pressure to certain lumens. The signal can also be used by the control system 124 to cause the irrigation fluid source 118 to turn on and/or to cause the valve 116 (via the valve controller 132) to fluidly couple certain lumens to the irrigation fluid source 118.

In response to the signal indicating a vacuum seal, the control system 124 can initiate supply of ablation energy to the ablation electrode 110 (step 410). In some embodiments, the signal indicating a vacuum seal initiates an alert, which may be an audible alert or a visual alert displayed on the display 140. As such, the control system 124 can cause the ablation energy generator 138 to turn on automatically or in response to input from an operator. The ablation electrode 110 can direct ablation energy to the tissue to form a lesion while stable contact between the catheter tip 106 and tissue is maintained via the vacuum seal.

The vacuum source 114 (along with the valve 116) can provide negative pressure for a predetermined period of time or until an operator provides input to release the catheter tip 106 from contact with the tissue. The catheter tip 106 can then be moved to an adjacent portion of tissue and various steps of the routine 400 can be repeated. If one or more of the sensors 122 detects a loss of the vacuum seal, the ablation energy can be stopped and/or an alert (e.g., audible or visual) can be initiated to let the operator know of the loss of the vacuum seal (step 412).

The control system 124 can include a computer-readable recording medium or “memory” 126 for storing processor-executable instructions, data structures and other information. The memory 126 may comprise a non-volatile memory, such as read-only memory (ROM) and/or flash memory, and a random-access memory (RAM), such as dynamic random access memory (DRAM), or synchronous dynamic random access memory (SDRAM). In some embodiments, the memory 126 may store processor-executable instructions that, when executed by a processor 128, perform routines for carrying out the functions related to maintaining stable contact between a catheter and tissue during ablation.

In addition to the memory 126, the control system 124 may include other computer-readable media storing program modules, data structures, and other data described herein for assessing and monitoring tissue ablation. It will be appreciated by those skilled in the art that computer-readable media can be any available media that may be accessed by the control system 124 or other computing system for the non-transitory storage of information. Computer-readable media includes volatile and non-volatile, removable and non-removable recording media implemented in any method or technology, including, but not limited to, RAM, ROM, erasable programmable ROM (EPROM), electrically-erasable programmable ROM (EEPROM), FLASH memory or other solid-state memory technology, compact disc ROM (CD-ROM), digital versatile disk (DVD), BLU-RAY or other optical storage, magnetic tape, magnetic disk storage or other magnetic storage devices and the like.

It will be appreciated that the structure and/or functionality of the control system 124 may be different than that illustrated in FIG. 1 and described herein. For example, the processor 128, measurement sub-unit 130, valve controller 132, mapping sub-unit 134, and display controller 136, and other components of the control system 124 may be integrated within a common integrated circuit package or distributed among multiple integrated circuit packages that together form control circuitry. It will be further appreciated that the control system 124 may not include all of the components shown in FIG. 1, may include other components that are not explicitly shown in FIG. 1 such as additional controllers dedicated to specific functions or steps in the routine 400, or may utilize an architecture different than that shown in FIG. 1.

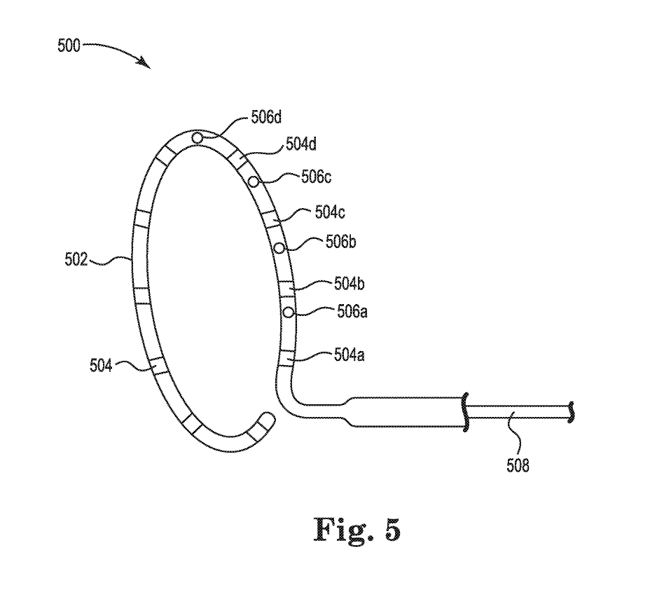

FIG. 5 shows a schematic view of another type of catheter 500 that can be used in the system 100. The catheter 500, with its loop-shaped catheter tip 502, is designed as a “single-shot” ablation catheter. The catheter tip 502 includes a plurality of ablation electrodes 504 and a plurality of openings 506 positioned around the catheter tip 502. FIG. 5 shows at least one opening 506 being positioned between a pair of ablation electrodes 504. At least one lumen 508 is coupled to the plurality of openings 506 and extends through the catheter 500.

The catheter 500 of FIG. 5 can be utilized to carry out various functions described with respect to routine 400. The catheter 500 can provide additional functionality because of the loop-shaped catheter tip 502 and the plurality of ablation electrodes 504. During an ablation procedure, once the catheter tip 502 is advanced to a desired ablation site, a negative pressure can be provided to at least one lumen 508 via the vacuum source 114 and the valve 116. In some embodiments, the control system 124 first causes a negative pressure to be generated at a first opening 506a only. Once it is determined that a vacuum seal has developed at the first opening 506a, a first ablation electrode 504a can be energized until the targeted section of tissue is ablated. Next, the control system 124 can cause a negative pressure to be generated at a second opening 506b only, determine that a vacuum seal has developed, and energize a second ablation electrode 504b. Such a sequence can be repeated for a third opening 506c and third ablation electrode 504c and so on (e.g., 506d, 504d) until a desired lesion has been created. In other embodiments, multiple ablation electrodes 504 are energized upon determining that a vacuum seal has developed. In other embodiments, the control system 124 can cause a negative pressure to be generated at multiple openings simultaneously.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A catheter system comprising:
a catheter including:
an elongated catheter body,
a first lumen extending through the elongated catheter body and configured to provide a vacuum,
a second lumen extending through the elongated catheter body and configured to provide a vacuum, and
a catheter tip coupled to the elongated body, wherein the catheter tip includes a first opening coupled to the first lumen and configured to provide a vacuum seal and a second opening coupled to the second lumen and configured to provide a vacuum seal.

2. The catheter system of claim 1, further comprising:
a valve coupled to the first and second lumens, wherein the valve is configured to couple the first and second lumens to a vacuum source.

3. The catheter system of claim 2, wherein the valve is further configured to couple the first and second lumens to an irrigation fluid source.

4. The catheter system of claim 1, further comprising:
a third lumen extending through the elongated catheter body and configured to provide a vacuum, and
wherein the catheter tip includes a third opening coupled to the third lumen and configured to provide a vacuum seal.

5. The catheter system of claim 1, wherein the catheter tip includes a plurality of ablation electrodes, and wherein the first opening is positioned between a pair of the plurality of ablation electrodes.

6. The catheter system of claim 1, wherein the catheter tip further includes a temperature sensor.

7. The catheter system of claim 6, wherein the catheter tip further includes at least one mapping sensor.

8. The catheter system of claim 1, wherein the first opening and the second opening have a surface area of 1-7.5 mm².

9. A catheter system comprising:
control circuitry configured to:
initiate a source of vacuum pressure to one or more lumens,
receive an indication of a vacuum seal, and
in response to the indication of the vacuum seal, initiate a source of ablation energy.

10. The catheter system of claim 9, wherein the control circuitry is configured to initiate the source of ablation in response to calculating that the vacuum seal is maintained for a predetermined period of time.

11. The catheter system of claim 9, wherein the control circuitry is further configured to:
receive an indication of contact of an ablation tip, and
in response to receiving the indication of contact, initiate the source of vacuum pressure.

12. The catheter system of claim 9, wherein the control circuitry is further configured to:
receive an indication of a loss of a vacuum seal, and
in response to receiving the indication of a loss of a vacuum seal, turn off the source of ablation energy.

13. The catheter system of claim 9, wherein the control circuitry is further configured to:
receive an indication of a loss of a vacuum seal, and
in response to receiving the indication of a loss of a vacuum seal, initiate an alert.

14. The catheter system of claim 9, wherein the control circuitry is configured to control a valve coupled to a first lumen and a second lumen, the control system further configured to:
cause the valve to fluidly couple the first lumen to the source of vacuum pressure, and
cause the valve to fluidly couple the second lumen to a source of irrigation fluid.

15. The catheter system of claim 9, wherein the control circuitry is configured to control a valve coupled to a plurality of lumens, the control system further configured to:
cause the valve to fluidly couple only one of the plurality of lumens to the source of vacuum pressure.

16. The catheter system of claim 9, further comprising:
a sensor configured to detect a vacuum seal.

17. The catheter system of claim 16, wherein the vacuum seal is detected by one of a pressure change and a change in blood flow.

18. The catheter system of claim 17, wherein the sensor is one of an optical sensor, a flow sensor, a pressure sensor, and an oxygen sensor.

19. The catheter system of claim 9, wherein the source of vacuum pressure is configured to provide at least 5 grams of force.

20. The catheter system of claim 9, wherein the control circuitry is configured to maintain the vacuum seal while ablation energy is applied.
